(19) United States
(12) Patent Application Publication
(10) Pub. No.: US 2006/0042627 A1
(43) Pub. Date: Mar. 2, 2006

(54) PAP titrate control method and apparatus

(76) Inventors: Nicholas J. MacMillan, Greensburg, PA (US); Michael B. Knepper, Friedens, PA (US)

Correspondence Address: MACMILLAN SOBANSKI & TODD, LLC;ONE MARITIME PLAZA FOURTH FLOOR, 720 WATER STREET, TOLEDO, OH, 43604-1619, US

(21) Appl. No.: 10/930,607

(22) Filed: Aug. 31, 2004

(51) Int. Cl. A61M 16/00

(52) U.S. Cl. 128/204.22; 128/204.21; 128/204.18

(57) ABSTRACT

A PAP device is provided with a programmed controller that is operative to incrementally increase the pressure of air supplied by the PAP device at predetermined time intervals. during a PAP titration.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO A SEQUENCE LISTING, A TABLE, OR A COMPUTER PROGRAM LISTING COMPACT DISC APPENDIX

Not Applicable

### BACKGROUND OF THE INVENTION

This invention relates in general to Polysomnogram testing and in particular to a PAP titration apparatus and method.

Many sleep related respiratory conditions are caused by a blockage or partial blockage of the respiratory tract. Snoring, for example, is caused by a partial blockage of the respiratory tract. As the blockage increases, hypopnea or a reduction of air flow to the lungs occurs. Apnea, or a temporary cessation of breathing, can occur when the airway becomes totally blocked. A person suffering from sleep apnea may have difficulty functioning during the day because of insufficient sleep caused by the apnea events. In severe cases, the person also can suffer from problems caused by reduced blood oxygen levels.

One form of treatment for severe snoring, hypopnea and sleep apnea involves the application of a pneumatic splint to the person's respiratory tract while he sleeps. Sufficiently high Positive Airway Pressure (PAP) is applied to the person's airway to prevent its collapse or blockage. The applied positive pressure is usually within a range of three to 20 cm H₂O. The positive air pressure is supplied by a PAP device that includes a regulated blower that supplies a flow of air to a face mask via a flexible tube. The face mask is designed to cover the user's nose and/or mouth and forms an air-tight seal with the user's face. Typically, when the PAP device is initially turned on, the device supplies a comfortable lower pressure to the person while he falls asleep. The PAP device then gradually increases the pressure to a prescribed therapeutic level.

A physician who suspects that a person is suffering from one of the above-described sleep disorders will refer the person to a sleep clinic for a sleep study, or Polysomnogram (PSG), to determine the exact nature of the problem and to select the appropriate treatment. A PSG is usually a multi-component test. The first component involves an overnight diagnostic test during which the patient's sleep is monitored. Referring now to FIG. 1, there is illustrated a schematic diagram of such a test. The subject 10 being tested is placed in a sleeping room 12. A plurality of sensors 14, three of which are shown in the figure, are attached to the subject 10. Sensors attached to the subject's head detect brain waves and muscle activity, such as face twiches and teeth grinding. Additionally, eye movements, such as Rapid Eye Movement (REM) are also detected by sensors placed adjacent to the subject's eyes. Electrocardiogram sensors detect heart activity while chest and abdomen belts detect breathing depth, apnea and hypopnea events. An oximeter (not shown) attached to a fingertip detects blood oxygen saturation. Sensors placed upon the subject's legs monitor leg movement during sleep. Finally, a video camera 16 monitors the subject's sleep throughout the night.

The sensors are connected to a Terminal Box, or “Pin Box” 18 located in the sleeping room 12. The Terminal Box 18 provides connection terminals for the sensor wires and is connected by cables 20 to a remotely located monitoring station 22. The Terminal Box may be temporally disconnected form the cables 20 to allow the subject mobility within the sleeping room 12. The monitoring station 22, which is manned throughout the PSG diagnostic test by a polysonmographic technician, includes a PSG subsystem 24 that receives and records all the data provided by the sensors 14. The subsystem 24 is typically a personal computer and is monitored and controlled by the polysonmographic technician with a keyboard 26 and display monitor 28. Additionally, a printer (not shown) for downloading data and a loudspeaker (not shown) for monitoring the subject 10 may be provided.

If, upon review of the diagnostic test results, the physician determines that the subject is suffering from Obstructive Sleep Apnea (OSA), he will order the second part of the PSG which involves another overnight stay at the sleep clinic to determine an appropriate pressure to overcome the airway obstruction. A schematic diagram of the apparatus for the second part is shown in FIG. 2, where components that are the same as shown in FIG. 1 have the same numerical identifiers. The same sensors 14 used during the diagnostic test are again attached to the subject 10. Additionally, the subject 10 is fitted with a nasal breathing mask 40. The mask is connected by a flexible air tube 42 to a PAP device 44 that is operative to supply a flow of pressurized air via the tube 42 and mask 40 to the subject's respiratory tract. The PAP device 44 is connected to an Analog Break Out Box (ABOB) 45 by a line 46. The ABOB 45 is, in turn, connected to the PSG subsystem 24 by a line 48. The ABOB 46 allows adjustment of the actual pressure of the air supplied to the subject 10 from the monitoring station 22 by the polysonmographic technician. Additionally, the ABOB 45 is connected by a data line 49 to the pin box 18 to provide operating data, such as pressure and flow rate, from the PAP device 44 to the PSG subsystem 24 for recodation.

During the first part of the night, the polysonmographic technician carries out a PAP titration during which the pressure of the air supplied to the subject 10 by the PAP device 44 is progressively increased. Typically, the pressure is increased in increments of 0.5 cm H₂O from an initial starting pressure of three cm H₂O. After each incremental increase, the monitored subject data is reviewed for a period of time before applying the next incremental increase. From a comparison of the data, an optimal pressure setting is selected and applied to the subject 10 for the remainder of the night. For ongoing treatment, a PAP apparatus is calibrated with the optimal pressure setting and presented to the subject 10 for his use.

It is apparent from the above description that the polysonmographic technician is very busy during the PAP titration. The technician not only has to continuously evaluate the sensed data, but he also must remember to periodically adjust the pressure of the air being supplied by the PAP apparatus 22. Accordingly, it would be desirable to to simplify the titration process to aid the technician.

### BRIEF SUMMARY OF THE INVENTION

This invention relates to an improved PAP titration apparatus and method.

The present invention contemplates a PAP device that is operative to supply a flow of pressurized air at a selected pressure. The invention also includes a programmed controller connected to the PAP device that is operative to increase the pressure of air supplied by the PAP device by a predetermined increment at predetermined time intervals.

The present invention also contemplates a method for titrating a PAP device that includes providing a programmed controller connected to a PAP device that is operative to incrementally increase the pressure of air supplied by the PAP device at predetermined time intervals. The controller is the actuated and the operation of the PAP device is monitored.

Various objects and advantages of this invention will become apparent to those skilled in the art from the following detailed description of the preferred embodiment, when read in light of the accompanying drawings.

### BRIEF DESCRIPTION OF THE DRAWINGS

FIG. 1 is a schematic diagram of a known apparatus for a diagnostic polysomnogram.

FIG. 2 is a schematic diagram of a known apparatus for a PAP titration.

FIG. 3 is a schematic diagram of an apparatus for PAP titration in accordance with the present invention.

FIG. 4 is a flow chart for a method of operating the apparatus shown in FIG. 2.

FIG. 5 is a flow chart of an enhanced version of the method shown in FIG. 4.

FIG. 6 is an alternate embodiment of the flow chart shown in FIG. 5.

### DETAILED DESCRIPTION OF THE PREFERRED EMBODIMENT

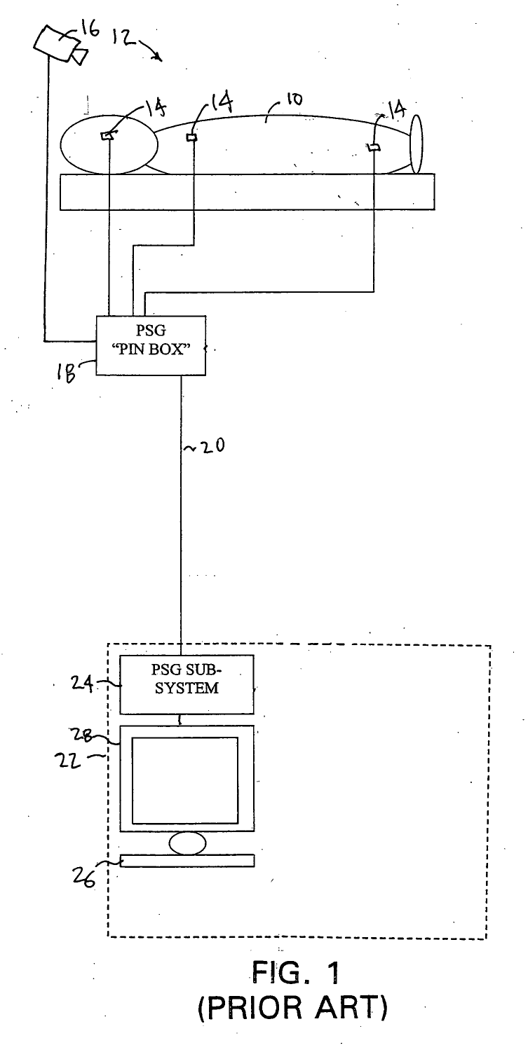

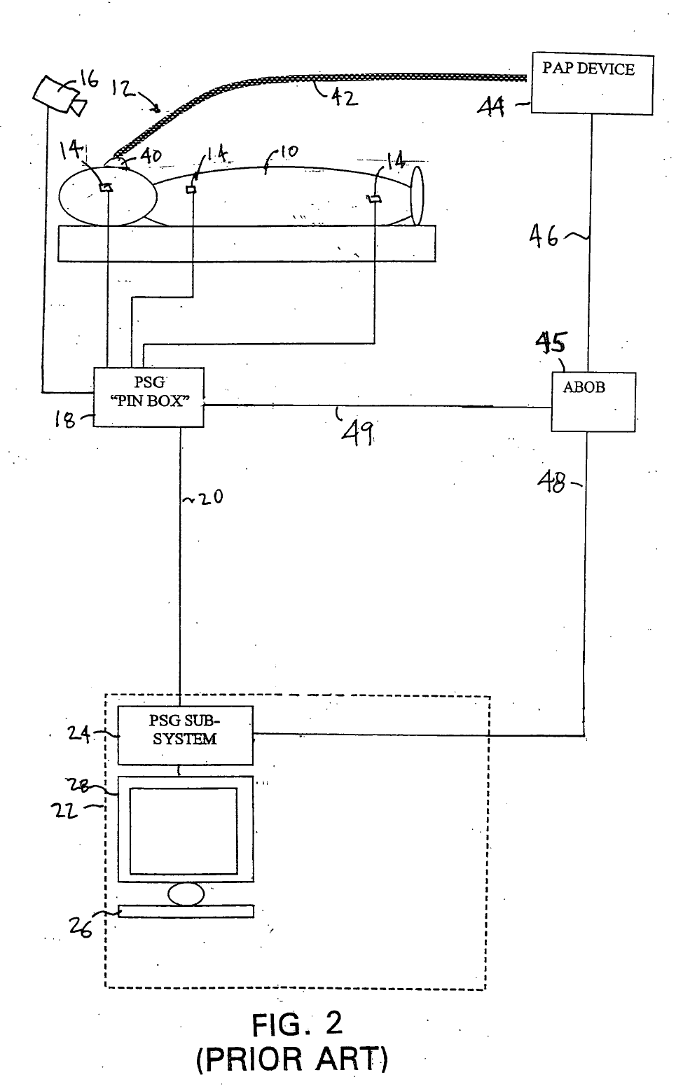

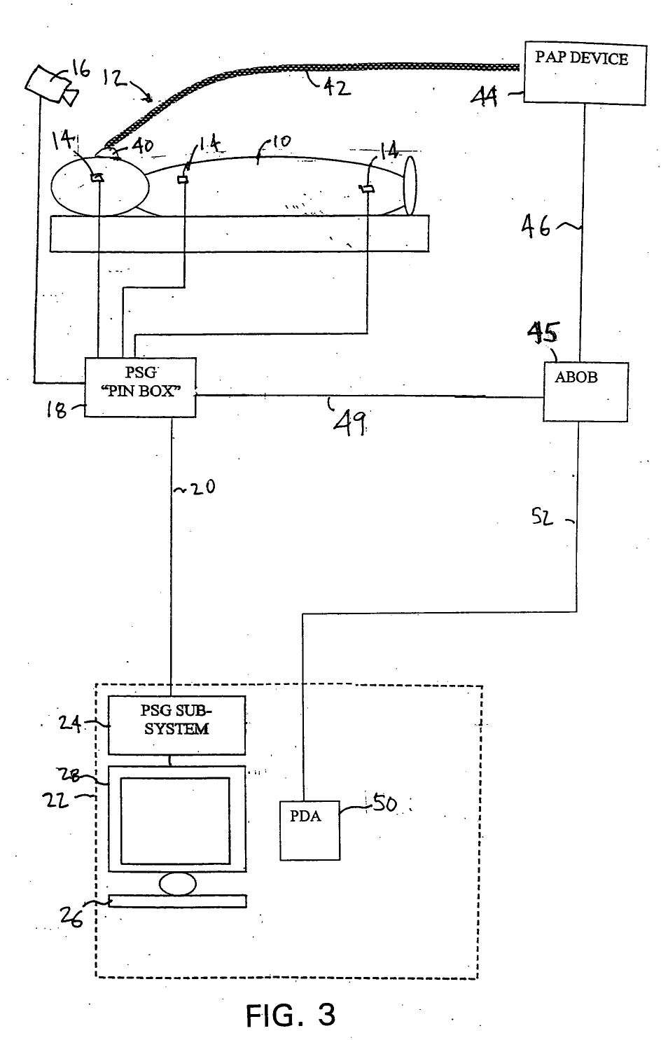

Referring again to the drawings, there is illustrated in FIG. 3 a schematic diagram of an apparatus for PAP titration in accordance with the present invention. Components shown in FIG. 3 that are similar to components shown in FIGS. 1 and 2 have the same numerical identifiers. The present invention contemplates providing a programmed Personal Digital Assistant (PDA) 50 that is either hardwired, or connected by wireless methods, to the ABOB 45 and is operative to titrate the PAP device 44. As shown in FIG. 3, the PDA is located at the monitoring station 22 connected to the ABOB 45 by the line 52. While the PDA 50 is illustrated as being located at the monitoring station 22, it will be appreciated that the PDA 50 also may be included in the PAP device 44 (not shown) or the within the ABOB 45 (not shown).

The PDA 50, also commonly referred to as a hand-held computer, is a handheld device that typically combines computing, telephone/fax, internet and networking features. Most PDA's use a stylus instead of a keyboard for input. Thus, the PDA 50 is actuated with either a stylus or its keyboard, depending upon the specific device. The invention also contemplates that the PDA 50 may be hardwired or connected by a wireless method to the PSG subsystem 24. In this case, the technician could use the keyboard 26 to actuate the PDA 50. In the preferred embodiment, any one of a number of hand held computers may be utilized, such as, for example, Hand Spring, Palm, Visor or Clié; however, the invention also may be practiced with other PDA's than those named.

The invention contemplates that the polysonmographic technician programs the PDA 50 with a “titrate assist” convenience feature by setting incremental pressure changes, time intervals and a maximum pressure. When the titrate assist feature is activated, the PDA 50 first verifies that it is actually connected to an ABOB 45. Typically, only sleep clinics are equipped with ABOB's. The titrate assist continues only upon verification that the PDA 50 is connected to a ABOB 45. Thus, the verification confirms that the PDA is being used for its intended function. Following verification of the presence of the ABOB 45, the PDA 50 is operative to place the PAP device 44 in the PAP mode. Then a PDA based algorithm that is stored in the PDA 50 begins sending step-wise incremental pressure increase commands to the PAP device 44 at the preset set time intervals. The PDA 50 continues to increase the pressure until the predetermined maximum pressure limit is sent. Thus, the PDA 50 becomes a timer and provider of pressure set signals to the PAP device 44. When the PDA 50 determines that it has sent the maximum permitted pressure set signal, it ceases sending signals. As indicated above, the starting pressure, pressure increments, time intervals and the maximum pressure limit are pre-set by the polysonmographic technician before the start of the test.

In the preferred embodiment, the incremental pressure step increases can vary from 0.5 cm H₂O to 5 cm H₂O in 0.5 cm H₂O pressure adjustment increments; however, the invention also may be practiced with other sizes of pressure step increases and pressure adjustment increments. Additionally, in the preferred embodiment, the time interval between pressure step changes can vary from one minute to 30 minutes in one minute time adjustment increments. Again, the invention also may be practiced with other time period intervals and time adjustment increments.

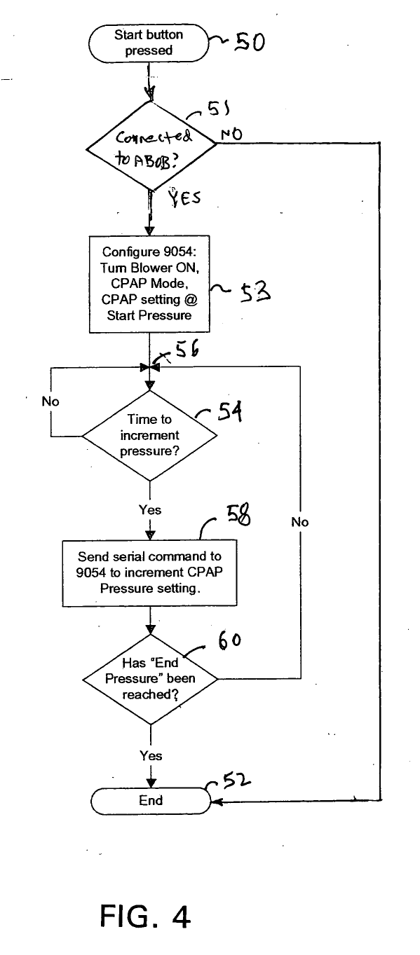

The present invention also contemplates a method for operation of the PDA 50 that is described by the PDA algorithm. A flow chart for the PDA algorithm is shown in FIG. 4. The procedure starts with block 60 when the polysonmographic technician depresses a start button on either the PDA 50 or his control panel. The algorithm advances to decision block 51 where the PDA 50 verifies that it is connected to the ABOB 45. If the PDA 50 is not connected to the ABOB 45, the algorithm transfers to end block 52 and halts operation. If the PDA 50 verifies the connection to the ABOB 45, the algorithm transfers to functional block 53 where the initial conditions are set for the PAP device 44. As shown in FIG. 4, the device blower is turned on, the PAP mode is selected and the starting pressure is set. Additionally, the PDA software is started to initialize an iteration counter as an internal timer. The algorithm then advances to decision block 54 where the timer is checked. If the predetermined time interval has not elapsed, the algorithm transfers to point 56 and continues. If, in decision block 54, the predetermined time interval has elapsed, the algorithm transfers to functional block 58 and the PDA 50 sends a serial command to the PAP device 44 to incrementally increase the pressure of the air being supplied to the subject 10. The algorithm then advances to decision block 60 where the PDA 50 determines whether the “end” pressure has been reached. If the end pressure has been reached, the algorithm transfers to end block 52 and halts operation, leaving the PAP device 44 supplying air at the end pressure. If the end pressure has not been reached, the software resets the internal PDA timer and the algorithm transfers to point 56 and continues as described above.

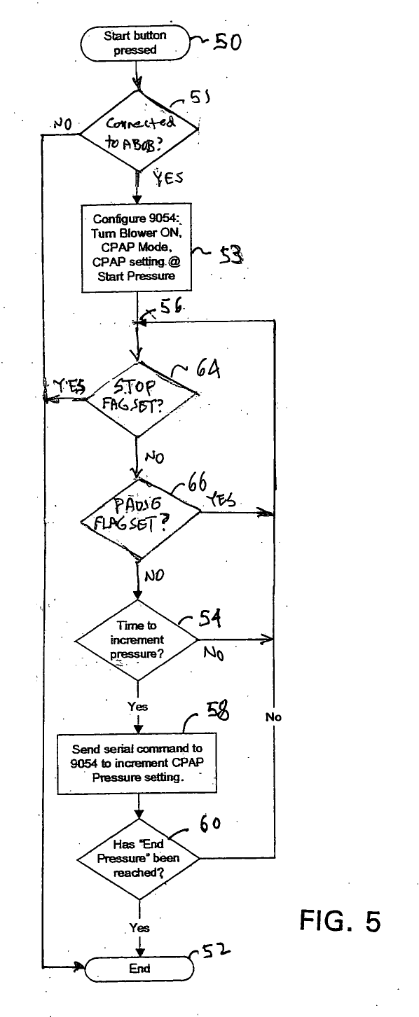

The invention also contemplates that the PDA 50 may include pause and stop features that allow the polysonmographic technician to hold the current pressure for the duration of the pause or to stop the process. The flow chart shown in FIG. 5 includes these features. Blocks shown in FIG. 5 that are similar to blocks shown in FIG. 4 have the same numerical identifiers. As can be seen, the flow chart in FIG. 5 includes two additional decision blocks that are numbered 64 and 66. The invention contemplates that the controls at the monitoring station 22 include pause and resume buttons (not shown) which may be included on either the PDA 50 or the keyboard 26. When the pause button is depressed by the polysonmographic technician, a PAUSE FLAG is set in the algorithm. Similarly, when the resume button is depressed, the PAUSE FLAG is reset, or cleared. The monitoring station 22 would also include a stop button for ending the test. When the stop button is depressed by the polysonmographic technician, a STOP FLAG is set in the algorithm. As shown in FIG. 5, after the initial conditions are set in functional block 52 and during every iteration of the PDA 50, decision block 64 is reached. In decision block 64, the STOP FLAG status is checked. If the STOP FLAG is set, the algorithm transfers to the end block 52 and halts operation, leaving the PAP device 44 supplying air at the last pressure setting. Thus, the STOP FLAG provides a system interrupt function. If the STOP FLAG is not set in decision block 64, the algorithm transfers to decision block 66. Once the STOP FLAG is set, the system may be subsequently restarted by pressing the start button in block 50 which would cause all of the variables to be reset in functional block 53, to include the STOP FLAG and the PAUSE FLAG (not shown).

In decision block 66, the PAUSE FLAG status is checked. If the PAUSE FLAG is set, the algorithm transfers to point 56 and continues operation, leaving the PAP device 44 supplying air at the current pressure setting. If the PAUSE FLAG is not set in decision block 66, the algorithm transfers to decision block 54 and continues as described above. Thus, as shown in the flow chart, the algorithm will remain in a loop until the PAUSE FLAG is reset. By checking the status of the STOP FLAG before checking the status of the PAUSE FLAG, the algorithm allows a system interrupt during a pause.

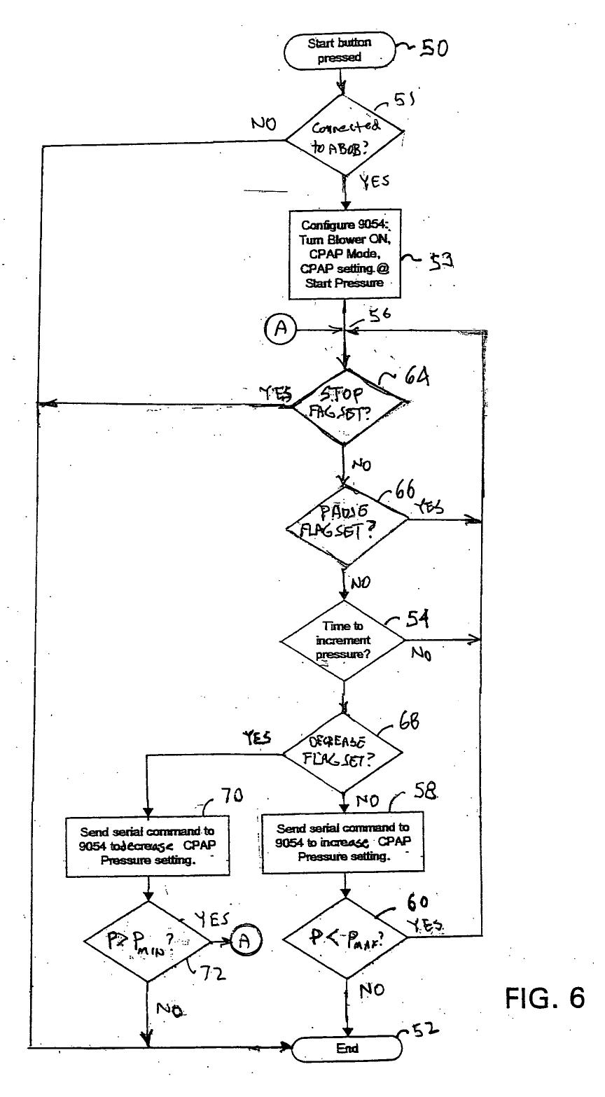

The invention also contemplates that the PDA 50 may include a pressure reduction feature that would allow the polysonmographic technician to incrementally reduce the pressure of the air supplied by the PAP device 44. Such a function may be desirable to allow the technician to “fine tune” the pressure supplied to the subject 10. The flow chart shown in FIG. 6 includes this feature. Blocks shown in FIG. 6 that are similar to blocks shown in FIGS. 4 and 5 have the same numerical identifiers. The feature is actuated by when the polysonmographic technician depresses a decrease button (not shown) that is either located upon the PAD 50 or the keyboard 26. Depressing the button sets a DECREASE FLAG. The invention also contemplates providing a decrease cancel button the would reset the DECREASE FLAG. As can be seen, the flow chart in FIG. 6 includes an additional decision block that is numbered 68 and is located immediately after the timer check decision block 54. Upon reaching the decision block 68, the algorithm checks the DECREASE FLAG. If the DECREASE FLAG is not set, the program transfers, as described above, to functional block 58 where the air pressure is incrementally increased. If, in decision block 68, the DECREASE FLAG is set, the algorithm transfers to functional block 70 where the air pressure is incrementally decreased. In the preferred embodiment, the magnitude of the incremental decrease is the same as the magnitude of the incremental increase provided in functional block 53. However, the invention also contemplates that a magnitude for the incremental decrease that is different from the magnitude of the incremental increase may be input in functional block 53 (not shown). The algorithm then advances to functional block 72 where the pressure of the air being supplied by the PAP device 44 is compared to a minimum pressure, Pₘᵢₙ. In the preferred embodiment, the minimum pressure is the starting pressure; however, the invention also contemplates that a predetermined minimum pressure may be set in functional block 53 (not shown). If, in decision block 72, the pressure is greater than the minimum pressure Pₘᵢₙ, the algorithm transfers back to point 56 and continues as described above. If, in decision block 72, the pressure is less than, or equal to, the minimum pressure Pₘᵢₙ, the algorithm transfers to end block 52 and terminates. At any time before the minimum pressure Pₘᵢₙ is reached, the polysonmographic technician may reset the DECREASE FLAG, in which case the algorithm will return to incrementally increasing the pressure. Similarly, the polysonmographic technician may pause or stop the algorithm at any time, as described above.

Also shown in FIG. 6 is an alternate representation of pressure check in decision block 60. In decision block 60, the pressure of the air being supplied by the PAP device 44 is compared to a maximum pressure, Pₘₐₓ, that is set in functional block 53. If the pressure is less than the maximum pressure Pₘₐₓ, the algorithm transfers back to point 56 and continues as described above. If, in decision block 60, the pressure is greater than, or equal to, the maximum pressure Pₘₐₓ, the algorithm transfers to end block 52 and terminates.

It will be appreciated that, while a button is described above for actuating the stop, pause and decrease features, depending upon the specific PDA 50 used, a stylus may be used in place of a button.

The titration method described above is an open loop process, in that it provides input signals for pressure change to the PAP device 44 on a timed basis. The PDA 50 makes no therapy changes as a result of observing the response of the subject to the changed pressure. Additionally, the PDA 50 does not interpret any resulting subject data. These functions remain under the control of the polysonmographic technician or other sleep clinic personnel. Furthermore, the PDA 50, as illustrated does not change any portion of the PAP device 44. Additionally, it will be appreciated that the flow charts shown in FIGS. 4 and 5 are intended to exemplary and that the invention also may be operated with other algorithms than are shown in the figures.

While the preferred embodiment of the invention has been described and illustrated as including a PDA 50, it will be appreciated that the invention also may be practiced with other devices, such as, for example, a microprocessor that has been programmed with one of the algorithms described above. Alternately, the personal computer included in the monitoring station 22 may be programmed with one of the algorithms described above to incrementally increase the pressure supplied by the PAP apparatus 44 at predetermined time increments. Additionally, as described above, the invention contemplates that communication between the various devices may be either hardwired or use wireless technology.

In accordance with the provisions of the patent statutes, the principle and mode of operation of this invention have been explained and illustrated in its preferred embodiment. However, it must be understood that this invention may be practiced otherwise than as specifically explained and illustrated without departing from its spirit or scope.

What is claimed is:

1. A PAP titration device comprising:
a PAP device, said PAP device operative to supply a flow of pressurized air at a selected pressure; and
a programmed controller connected to said PAP device, said controller being operative upon command to increase the pressure of air supplied by said PAP device by a predetermined pressure increment at predetermined time intervals.

2. The device according to claim 1 wherein said controller is adapted to receive and store values for said predetermined incremental pressure increase and said predetermined time interval before a command to increase pressure is received.

3. The device according to claim 2 wherein said controller is connected to said PAP device through an analog breakout box and further wherein said controller is operative, upon receiving said command to increase pressure, to confirm that said connection through said analog breakout box exists and to only begin increasing the pressure upon confirmation of said connection.

4. The device according to claim 3 wherein said controller is located remotely from said PAP device.

5. The device according to claim 4 wherein said controller is hardwired to said analog breakout box.

6. The device according to claim 4 wherein said controller has a wireless connection to said analog breakout box.

7. The device according to claim 4 wherein said controller is a programmed personal data assistant.

8. The device according to claim 4 wherein said controller is programmed microprocessor.

9. The device according to claim IC wherein said controller is a programmed personal computer.

10. The device according to claim 2 wherein said controller is further operative upon command to decrease the pressure of air supplied by said PAP device by a predetermined pressure increment at predetermined time intervals.

11. The device according to claim 2 wherein said controller is further operative upon command to hold the pressure of air supplied by said PAP device at a constant pressure.

12. The device according to claim 2 wherein said controller limits the pressure increases such that the pressure of air supplied by said PAP device is less than a predetermined maximum pressure.

13. The device according to claim 12 wherein said controller is adapted to receive and store a value for said predetermined maximum pressure before a command to increase pressure is received.

14. A method for titrating a PAP device comprising the steps of:
(a) providing a programmed controller connected to a PAP device, the controller being operative to incrementally increase the pressure of air supplied by the PAP device at predetermined time intervals;
(b) actuating the controller with the controller causing the PAP device to increase the pressure of the air supplied by the pressure increment after each passage of the predetermined time increment; and
(c) monitoring the effects of the operation of the PAP device.

15. The method according to claim 14 wherein, prior to step (b), the controller receives and stores values for the predetermined incremental pressure increase and the predetermined time interval used in step (b).

16. The device according to claim 15 wherein the controller supplied in step (a) is connected to the PAP device through an analog breakout box and further wherein during step (b), after actuation, but before the first incremental pressure increase, the controller is operative to confirm that the connection through the analog breakout box exists before beginning proceeding.

17. The device according to claim 16 wherein the controller is located remotely from the PAP device.

18. The method according to claim 17 wherein the controller provided in step (a) is a programmed personal data assistant.

19. The method according to claim 17 wherein the controller provided in step (a) is a programmed microprocessor.

20. The method according to claim 17 wherein the controller provided in step (a) is a programmed personal computer.

21. The method according to claim 18 wherein the controller is further operative upon command to decrease the pressure of air supplied by said PAP device by a predetermined increment at predetermined time intervals.

22. The method according to claim 18 wherein the controller is further operative upon command to hold the pressure of air supplied by said PAP device at a constant pressure.

23. The method according to claim 18 further including, during step (b) comparing the supplied pressure to a predetermined maximum pressure controller and halting the pressure increases before the pressure of air supplied by said PAP device exceeds the predetermined maximum pressure.

24. The device according to claim 23 wherein, prior to step (b), the controller receives and stores a value for the predetermined maximum pressure.
